(19) United States
(12) Patent Application Publication
(10) Pub. No.: US 2026/0007407 A1
(43) Pub. Date: Jan. 8, 2026

(54) PULMONARY SHUNT SYSTEMS

(71) Applicant: EDWARDS LIFESCIENCES CORPORATION, Irvine, CA (US)

(72) Inventor: Arvin T. Chang, Yorba Linda, CA (US)

(21) Appl. No.: 19/325,334

(22) Filed: Sep. 10, 2025

Related U.S. Application Data
(63) Continuation of application No. PCT/US2024/020019, filed on Mar. 14, 2024.
(60) Provisional application No. 63/492,185, filed on Mar. 24, 2023.

(51) Int. Cl. A61B 17/11; A61F 2/848; A61F 2/95

(52) U.S. Cl. CPC A61B 17/11; A61F 2/95; A61B 2017/1107; A61B 2017/1139; A61F 2002/8483; A61F 2210/0076

(57) ABSTRACT

A method may involve creating a first opening in a right pulmonary artery and a second opening in a superior vena cava. A shunt may be percutaneously delivered into the first opening and the second opening. A stent may be percutaneously delivered into the superior vena cava adjacent to the shunt. The stent can comprise a covering to at least partially prevent flow of blood from the shunt through the stent and/or may be at least partially flexible to allow the stent to bend away from walls of the superior vena cava in response to inflow of blood through the shunt. The stent may be sized to seal against the superior vena cava at a first end and a second end of the stent to trap blood between the stent and the walls of the superior vena cava.

### RELATED APPLICATION

This application is a continuation of International Patent Application No. PCT/US2024/020019, filed Mar. 14, 2024, which claims the benefit of U.S. Provisional Application No. 63/492,185, filed Mar. 24, 2023, the disclosures of which are hereby incorporated by reference in their entirety.

### BACKGROUND

The present invention relates generally to the field of medical devices and procedures. Pulmonary hypertension is a rapidly deteriorating vascular disease associated with high short-term mortality rates. A primary driver of disease progression is the increase in pulmonary arterial pressure due to a reduction in vascular compliance.

### SUMMARY

Some implementations of the present disclosure relate to a shunt system including a shunt configured to maintain a blood flow pathway between a first blood vessel and a second blood vessel, and a deformable device configured to elastically deform in response to blood flow through the shunt.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loudspeakers, headphones, pressure transducers, temperature transducers, or using any combination of suitable technologies.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 illustrates an example representation of a heart including indicators representing blood flow through the heart.

FIGS. 2A and 2B illustrate optional delivery methods for delivering one or more implants described herein.

FIG. 3A illustrates delivery of a shunt and/or fluid transfer/movement device into the SVC and/or RPA.

FIG. 3B illustrates delivery of a stent and/or similar device into the SVC and/or RPA.

FIG. 4A illustrates a default and/or first state of a shunting system prior to movement of blood out of the RPA into the SVC.

FIG. 4B illustrates a second state of the shunting system in which blood pressure in the RPA increases (e.g., during systole) a sufficient amount for excess blood to be pushed out of the RPA, through the shunt, and/or into the SVC.

FIG. 4C illustrates a third state of the shunting system in which the blood pressure in the RPA decreases from the increased amount of the second state such that pulling and/or suction is created to cause blood within the SVC to move back through the shunt and/or into the RPA.

FIG. 5 provides a side view of another example shunting system for shunting blood between a first blood vessel and/or chamber (e.g., the RPA) and a second blood vessel and/or chamber (e.g., the SVC) in accordance with one or more examples.

FIG. 6 illustrates an example compliance system for shunting, transferring, and/or exchanging fluid (e.g., blood and/or saline) between a first blood vessel and/or chamber (e.g., the RPA) and a second blood vessel and/or chamber (e.g., the SVC) in accordance with one or more examples.

FIG. 7 illustrates an example compliance system for shunting, transferring, and/or exchanging fluid (e.g., blood and/or saline) between a first blood vessel and/or chamber (e.g., the RPA) and a second blood vessel and/or chamber (e.g., the SVC) in accordance with one or more examples.

FIG. 8 provides a flowchart illustrating an example process for delivery of one or more shunt implant and/or shunt systems describe herein.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

### Overview

The following includes a general description of human cardiac anatomy that is relevant to certain inventive features and examples disclosed herein and is included to provide context for certain aspects of the present disclosure.

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

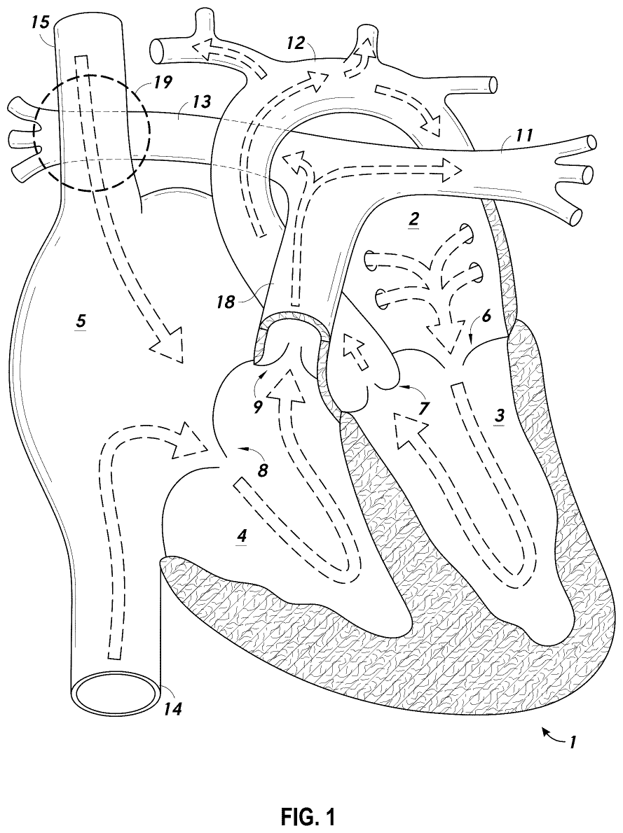

FIG. 1 illustrates an example representation of a heart 1 including indicators representing blood flow through the heart 1. The heart 1 includes four chambers, namely the left atrium 2, the left ventricle 3, the right ventricle 4, and the right atrium 5. A wall of muscle, referred to as the septum, separates the left 2 and right 5 atria and the left 3 and right 4 ventricles.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The valves of the heart 1 further include the pulmonary valve 9, which separates the right ventricle 4 from the pulmonary artery 18, and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant, and press back against the leaflets. As a result, the leaflets/cusps come in apposition to each other, thereby closing the flow passage.

Any of several access pathways may be utilized for maneuvering guidewires and catheters in and around the heart 1 to deploy medical implants (e.g., shunts) of the present application. For instance, access may be from above via either the subclavian vein or jugular vein into the superior vena cava (SVC) 15, right atrium (RA) 5. In another example, the access path may start in the femoral vein and through the inferior vena cava (IVC) 14 into the heart 1. Other access routes may also be used, and each typically utilizes a percutaneous incision through which the guidewire and catheter are inserted into the vasculature, normally through a sealed introducer, and from there the physician controls the distal ends of the devices from outside the body.

The pulmonary artery 18 branches into a right pulmonary artery (RPA) 13 and a left pulmonary artery (LPA) 11. Some examples of the present disclosure may involve delivering one or more implants (e.g., shunts) to an intersection 19 and/or crossing point between the RPA 13 and the SVC 15. For example, the RPA 13 may extend generally perpendicularly to the SVC 15 and/or can cross behind/in front of the SVC 15. In some cases, the RPA 13 may contact the SVC 15 while in other cases there may be a separation between the RPA 13 and the SVC 15.

Some examples described herein involve delivering a shunt and/or compliance system percutaneously to connect the RPA 13 with the SVC 15. Given that the RPA 13 and SVC 15 are adjacent anatomically, the intersection 19 area of the two provides an ideal location to establish a shunt. Further, because the RPA 13 has higher pressures than the SVC 15, particularly under pulmonary hypertensive conditions, unidirectional movement of blood flow is consistently diverted out of the RPA 13 and into the SVC 15. The net result of this shunting is to decompress and lower the pressure in the main pulmonary artery 18, including mean and peak systolic pressure. This is turn reduces the afterload on the right ventricle 4 and reduces the amount of work required to eject blood, thereby decreasing right ventricle 4 compensatory responses to pulmonary hypertension and preserving ventricular-vascular coupling. Movement (e.g., shunting, transfer, and/or exchange) of fluid (e.g., blood and/or saline) in some examples herein may occur at all pressures, or a shunt and/or compliance system may be pre-loaded to dynamically move fluid at an offset pressure. In some cases, the shunt and/or compliance systems may or may not lower pressure (e.g., in the pulmonary artery 18) significantly. The systems may be configured to allow the right ventricle 4 to eject more volume of blood during systole. The right ventricle 4 may experience the same peak pressure but may be enabled to eject more blood as the compliance chamber allows more capacity for blood flow.

Pulmonary hypertension is a rapidly deteriorating vascular disease associated with high short-term mortality rates. A primary driver of disease progression is the increase in pulmonary arterial pressure due to a reduction in vascular compliance. The reduction in compliance is caused by several key factors, namely remodeling of the microcirculation or arteriosclerosis due to elevated pressures or systemic inflammation, respectively. The consequence of sustained and progressive increases in pulmonary arterial pressure is right ventricular-vascular uncoupling, whereby the right ventricle is no longer able to compensate for the increase in afterload which is typically accomplished by increases in stroke volume and contractility. Once this uncoupling occurs, the right ventricle begins to dilate with increases in filling pressures that can lead to tricuspid regurgitation and peripheral venous congestion. The combination of reduced forward flow and increased backwards transmission of pressure results in a reduction of transpulmonary perfusion, loss of gas exchange, hypoxemia, impaired LV filling, venous congestion of peripheral organs, and ultimately cardiac failure. This invention seeks to reduce the pulmonary artery pressure (both mean pressure and systolic) which is associated with an increase in afterload and work performed by the right ventricle. Via reductions in pulmonary arterial pressure, examples of the present disclosure can preserve right ventricular function, attenuate the progressive remodeling that occurs, and/or prevent peripheral venous congestion and symptoms associated with poor transpulmonary perfusion. Some examples may be applicable across multiple types of pulmonary hypertensive conditions.

Examples of the present disclosure relate to various percutaneous shunting methods and/or shunting devices that may be delivered percutaneously to connect different blood flow pathways. While the disclosure herein focuses on connecting and/or shunting between the RPA 13 and the SVC 15, this is for illustrative purposes and the examples described herein can be applied to other areas of anatomy. Because the RPA 13 and SVC 15 are adjacent anatomically, the intersection point of the RPA 13 and the SVC 15 can provide an effective location to establish one or more shunts. Further, because the RPA 13 has higher pressures than the SVC 15, particularly under pulmonary hypertensive conditions, unidirectional movement of blood flow is consistently diverted out of the RPA 13 and into the SVC 15. Pressure in the RPA 13 can lower during diastole to allow SVC 15 pressure and/or the shunt and/or compliance systems described herein to push stored blood forward into the lungs. The net result of this shunting is to decompress and lower the pressure in the main pulmonary artery 18, including mean and peak systolic pressure. This is turn reduces the afterload on the right ventricle 4 and reduces the amount of work required to eject blood, thereby decreasing right ventricle 4 compensatory responses to pulmonary hypertension and preserving ventricular-vascular coupling. The various shunting methods described herein may be performed at any pressures and/or the various shunt devices described herein may be pre-loaded to dynamically shunt at an offset pressure.

The present disclosure provides methods and devices (including various medical implants) for shunting blood within a human body. The term “implant” is used herein according to its plain and/ordinary meaning and may refer to any medical implant, frame, valve, shunt, stent, anchor, and/or similar devices for use in treating various conditions in a human body. Implants may be delivered via catheter (i.e., transcatheter) for various medical procedures and may have a generally sturdy and/or flexible structure. The term “catheter” is used herein according to its broad and/ordinary meaning and may include any tube, sheath, steerable sheath, steerable catheters, and/or any other type of elongate tubular delivery device comprising an inner lumen configured to slidably receive instrumentation, such as for positioning within an atrium or coronary sinus, including for example delivery catheters and/or cannulas.

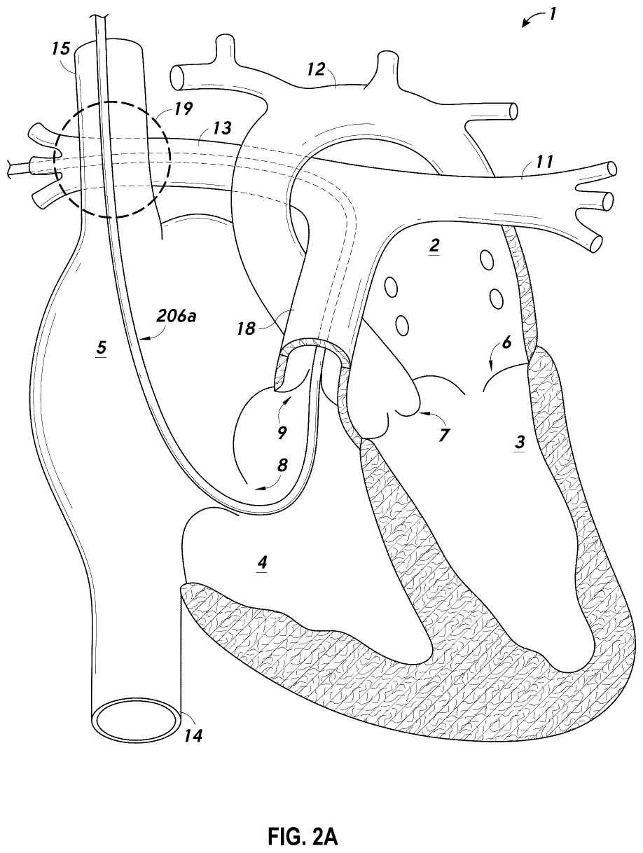

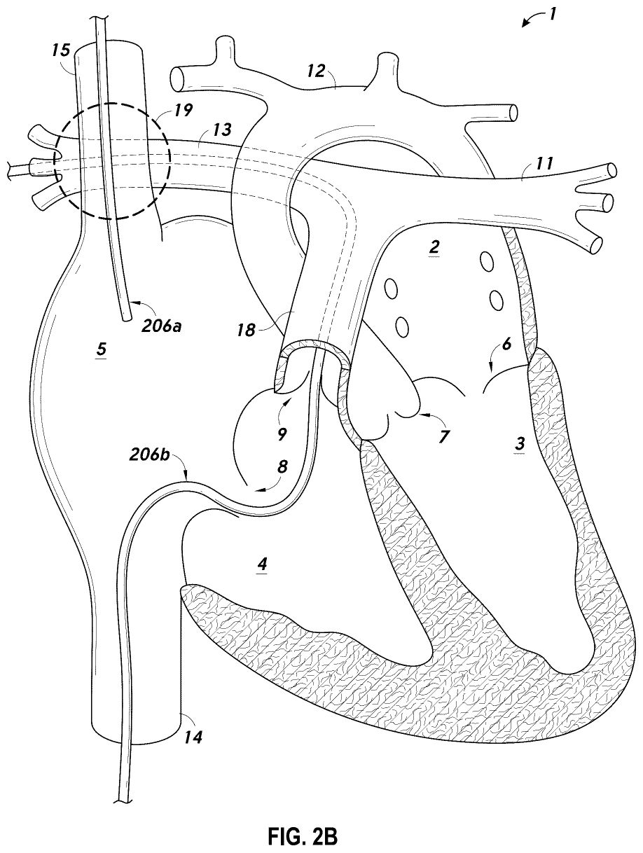

FIGS. 2A and 2B illustrate optional delivery methods for delivering one or more implants described herein. Some transcatheter processes described herein can utilize a single catheter 206 or multiple catheters 206. For example, FIG. 2A illustrates an example in which a first catheter 206a may be used to deliver one or more implants to the SVC 15 and/or the RPA 13. The first catheter 206a may be delivered to the SVC 15 and/or RPA 13 via any suitable delivery path. For example, the first catheter 206a may be delivered via the RPA 13 and the PA 18 and through the pulmonary valve 9 into the right ventricle 4, through the tricuspid valve 8 into the right atrium 5, and from the right atrium 5 into the SVC 15. Additionally or alternatively, the first catheter 206a may be delivered via the SVC 15 into the right atrium 5, through the tricuspid valve 8 into the right ventricle 4, through the pulmonary valve 9 into the PA 18 and finally into the RPA 13. Regardless of which delivery path is used, one or more implants may be delivered at the RPA 13 and/or SVC 15 at or near the intersection area 19.

FIG. 2B illustrates another example in which two catheters, a first catheter 206a and a second catheter 206b are delivered and/or used simultaneously. The first catheter 206a may be delivered via the SVC 15 and/or into the right atrium 5. The second catheter 206b may be delivered via the RPA 13 and the PA 18 and through the pulmonary valve 9 into the right ventricle 4, through the tricuspid valve 8 into the right atrium 5, and from the right atrium 5 out the IVC 14. Additionally or alternatively, the catheter 206 may be delivered via the IVC 14 into the right atrium 5, through the tricuspid valve 8 into the right ventricle 4, through the pulmonary valve 9 into the PA 18 and finally into the RPA 13. Regardless of which delivery path is used, one or more implants may be delivered at the RPA 13 and/or SVC 15 at or near the intersection area 19.

The one or more catheters 206 can be delivered via a transjugular, brachial, subclavian, and/or transfemoral approach. Within each access point, either one or two catheters 206 may be used for the delivery. Where two catheters 206 are used, the first catheter 206a may comprise a stent snare catheter and/or the second catheter 206b can comprise a puncture delivery catheter, or vice versa. In the case of a single catheter system, a puncture may be made and/or one or more implants delivered in one direction using the same catheter 206, in either SVC 15 to RPA 13 or vice versa directions. In some cases, it can be easier to deliver a catheter 206 through the RPA 13 than the SVC 15 and/or IVC 14.

The intersection area 19 between the RPA 13 and the SVC 15 may provide a suitable and/or desirable shunting location due at least in part to proximity and/or contact between the RPA 13 and SVC 15. In some cases, it may be advantageous to shunt from the RPA 13 to the SVC 15, particularly for patients experiencing pulmonary hypertension. For example, patients experiencing pulmonary hypertension can experience increases in pressure that can support the benefit of an inter-vasculature shunt. In some cases, the amount of disease in the pulmonary circulation can become independent of the left atrium. For example, even as left atrium pressure increases, pulmonary pressures can increase disproportionately to the left atrium pressure. As another example, even when left atrium pressure stabilizes, pulmonary pressure can continue to increase. Thus, an inter-vasculature shunt in a patient with pulmonary hypertension may provide incremental improvement, for example in comparison to unloading the pressure directly from the pulmonary circulation. By offloading pressure, right ventricle function can be improved. When the right ventricle starts to deteriorate, survival rate can decrease quickly. Any decrease in pressure, either locally or otherwise, can significantly reduce the amount of work the right ventricle has to perform to continue to pump blood to the left side of the heart.

The right side of heart (e.g., the right ventricle) may not be able to support relatively high loads. In contrast, the left side of the heart can be more suited to supporting large fluctuations of volume into itself. Thus, it can be beneficial to relieve pressure from the right side of the heart and instead moderately increase and/or redirect pressure to the left side.

A direction of flow between the RPA 13 and SVC 15 can be determined naturally. For example, a patient's condition (e.g., pulmonary hypertension) can cause the pressure in the RPA 13 to be higher, thus directing flow from the RPA 13 to the SVC 15.

In some cases, it may be advantageous to insert one or more delivery systems from the RPA 13 into the right ventricle 4 to allow for wiring in the direction of more length. This may be particularly advantageous in RPA 13/SVC 15 shunting because of the perpendicular orientation of the RPA 13 and SVC 15. One or more delivery systems may be maintained in the right ventricle 4 to provide trackability and support for other delivery system.

While certain example delivery methods are illustrated in FIGS. 2A and 2B, other delivery methods may be used for the example devices described herein. For example, a single catheter 206 may be delivered via the SVC 15 and/or the IVC 14. In another example, a first catheter 206a and/or a second catheter 206b may be delivered via the RPA 13 to the SVC 15 to allow one or more shunt devices to be delivered from the SVC 15 to the RPA 13. Additionally or alternatively, a first catheter 206a and/or a second catheter 206b may be delivered via the SVC 15 to the RPA 13 to allow one or more shunt devices to be delivered from the RPA 13 to the SVC 15.

### Pulmonary Shunt Systems

Examples described herein provide devices (e.g., medical implants) and/or methods (e.g., delivery methods) configured to reduce right ventricular afterload in patients with, for example, combined pre- and post-capillary pulmonary hypertension (CpcPH) due to left heart failure. Some examples may advantageously be configured to decrease progression to right ventricular failure. When a patient experiences increased afterload (e.g., right ventricle afterload), the heart (e.g., the right ventricle) may be required to overcome elevated pressure to eject blood to the lungs. Examples descried herein can advantageously reduce health complications in patients by reducing tissue wall stress (e.g., right ventricle walls) and/or reducing dilation (e.g., at the right ventricle).

The pulmonary and systemic circulation generally transport an equal amount of blood. The pulmonary circulation works at much lower pressures than the systemic circulation. Pulmonary pressure is generally lower because resistance is lower and the pulmonary vasculature is more compliant. The pulmonary artery with the two main branches account for only 15-20% of the total pulmonary arterial compliance, the rest being distributed throughout the entire pulmonary arterial vessels.

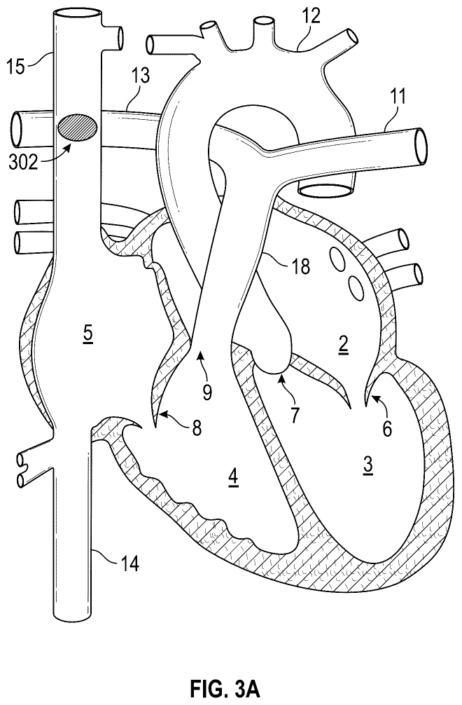

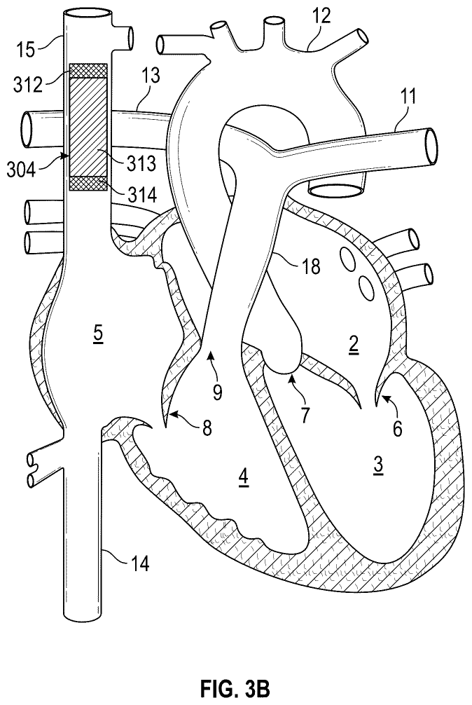

FIGS. 3A and 3B illustrate delivery of one or more implants and/or systems to an example heart 1 to increase compliance in accordance with one or more examples. While some examples herein may describe placement of one or more implants at the SVC 15 and/or RPA 13, this is for illustrative purposes and example implants may be configured for delivery and/or placement at other blood vessels and/or heart chambers.

FIG. 3A illustrates delivery of a shunt 302 and/or fluid transfer and/or exchange device into the SVC 15 and/or RPA 13. In some examples, the shunt 302 may be delivered via catheter. A puncture may be created in the SVC 15 and/or RPA 13 to accommodate the shunt 302. In some examples, the shunt 302 may be configured to extend at least partially into the SVC 15 and/or RPA 13 and/or across any gap between the SVC 15 and RPA 13. The shunt 302 may be configured to form a bridge between the SVC 15 and RPA 13 and/or may at least partially pull the SVC 15 towards the RPA 13 and/or the RPA 13 towards the SVC 15. The term “shunt” is used herein in accordance with its plain and ordinary meaning and may refer to any device and/or mechanism configured to allow for movement of fluid (e.g., blood and/or saline) from a first blood vessel and/or chamber to a second blood vessel and/or chamber and/or back from the second blood vessel and/or chamber to the first blood vessel and/or chamber. In some examples, fluid passed through the shunt 302 (e.g., from the RPA 13 to the SVC 15) may be returned through the shunt 302 (e.g., from the SVC 15 to the RPA 13). For example, the shunt 302 may comprise a bladder and/or inflatable body disposed in the SVC 15 and/or configured to fill with fluid from the RPA 13. The shunt 302 may be configured to press fluid back out of the bladder and/or inflatable body and/or into the RPA 13.

While the shunt 302 is described as bridging the SVC 15 and RPA 13, the shunt 302 and/or additional shunts may additionally or alternatively be used at other anatomical locations, including as a shunt between the RPA 13 and other venous side vessels and/or chambers (e.g., the right atrium).

In some examples, the shunt 302 may be at least partially fluid tight and/or may be configured to provide a channel for blood flow between the SVC 15 and RPA 13. For example, as blood pressure in the RPA 13 increases (e.g., during systole), blood may be pushed from the RPA 13 through the shunt 302 and/or to the SVC 15. As blood pressure in the RPA 13 decreases (e.g., during diastole), blood pushed into the SVC 15 from the RPA 13 may be pushed and/or suctioned back into the RPA 13 via the shunt 302.

The shunt 302 can have any suitable form and/or structure. In some examples, the shunt 302 may comprise a laser-cut hypotube and/or other device. The shunt 302 may have an at least partially cylindrical form and/or may comprise a lumen configured to allow blood flow through an outer frame of the shunt 302. In some examples, the shunt 302 may comprise flared ends (e.g., flanges) configured to facilitate anchoring of the shunt 302 against the walls of the SVC 15 and/or RPA 13.

The shunt 302 may be delivered to a point of the SVC 15 that is adjacent and/or near the RPA 13 and/or to a point of the RPA 13 that is adjacent to and/or near the SVC 15. For example, a puncture may be made and/or the shunt 302 may be delivered to a point of overlap between the SVC 15 and the RPA 13.

In some examples, the shunt 302 may be configured to form a connection and/or bridge between two or more blood vessels and/or chambers. The shunt 302 may be configured to be situated between two or more tissue walls and/or at least partially within at least one tissue wall. The shunt 302 may be configured to create and/or maintain a blood flow pathway between and/or through the tissue walls. The shunt 302 may be coupled to one or more anchoring mechanisms (e.g., flanges), which can include a distal anchoring mechanism and/or a proximal anchoring mechanism. The shunt 302 may form a generally tubular shape that can have a set/pre-formed size and/or a variable size.

The shunt 302 and/or anchoring mechanisms can be at least partially composed of any suitable material(s), which can include expandable stainless steel, cobalt chromium, textiles, and/or Nitinol. In some examples, the shunt 302 and/or anchoring mechanisms can be expanded via coaxial displacement of a delivery system (e.g., a catheter). The shunt effective orifice area (EOA) and/or diameter of the shunt 302 may be configured to support any desired amount of shunting. For example, the shunt 302 may be configured to achieve a minimum reduction in pulmonary pressure while preserving a transpulmonary pressure gradient required to facilitate pulmonary perfusion and delivery of blood to the left atrium. The shunt EOA and/or length of the shunt 302 may be configured to maintain a pressure reduction across a variety of clinical conditions, including but not limited to peripheral venous hypertension and exercise.

FIG. 3B illustrates delivery of a stent 304 and/or similar device into the SVC 15 and/or RPA 13. In some examples, the stent 304 may be delivered via catheter. The stent 304 may comprise one or more self-expanding and/or balloon-expandable materials and/or may be configured to naturally expand outwardly in the absence of substantial inward forces. In some examples, the stent 304 may be at least partially composed of one or more shape-memory alloys, which can include Nitinol and/or similar materials. The materials of the stent 304 may be shape-set in a generally open form. For example, the stent 304 may comprise an inner lumen that is fully open in the default form of the stent 304. The stent 304 may comprise walls and/or sides formed from sheets and/or lines of material that may be configured to bend inwardly in response to outward pressure (e.g., blood pressure).

The stent 304 may comprise a midsection 313 disposed between a first end 312 and/or a second end 314. The first end 312 and/or second end 314 may be configured to securely anchor to surrounding tissue and/or may be configured to resist deformation. The midsection 313 may be configured to be loosely anchored and/or detached from the surrounding tissue to allow the midsection 313 to bend inwardly in response to external pressure. In some examples, the stent 304 may be configured to be delivered adjacent to the shunt 302 and/or adjacent to a puncture point between the SVC 15 and/or RPA 13. For example, the midsection 313 may be configured to extend across an opening and/or channel formed between the SVC 15 and RPA 13.

In some examples, the stent 304 may be configured for delivery into the SVC 15, as shown in FIG. 3B. However, the stent 304 may be configured for delivery into the RPA 13 and/or other blood vessel and/or heart chamber.

The stent 304 may have a generally cylindrical shape and/or may have other suitable shapes. In some examples, the stent 304 may comprise a frame composed of one or more shape-memory alloys and/or generally rigid materials. The stent 304 may additionally comprise a covering and/or skirt at least partially enclosing an outer and/or inner surface of the frame. The covering may be at least partially fluid tight and/or may be configured to catch and/or retain blood pressed into contact with the covering. In some examples, the covering may be configured to extend along the midsection 313, first end 312 and/or second end 314 of the stent 304.

Blood passing through the shunt 302 (e.g., from the RPA 13) may press against the covering and/or may cause the midsection 313 of the stent 304 to bow inwardly. The first end 312 and/or second end 314 may be configured to retain a default form and/or may be configured not to bow inwardly in response to blood pressure changes. Accordingly, blood may be retained around the midsection 313 until the blood is pressed and/or pulled back through the shunt 302 (e.g., to the RPA 13). In some examples, the covering may not be fully fluid tight and/or may be configured to allow some blood flow through the covering to advantageously prevent clot and/or thrombus formation due to stagnant blood. Moreover, the first end 312 and/or second end 314 may be configured to form partial seals and/or may be configured to allow a controlled amount of blood to leak beyond the stent 304 and/or into the SVC 15.

The SVC 15 is a relatively large blood vessel. A significant level of compliance can be gained in the pulmonary arterial system by shunting blood back and forth into the SVC 15 from systole to diastole.

In some examples, the shunt 302 and/or stent 304 may be at least partially composed of braided materials, which may include stainless steel, Nitinol, and/or other metals, polymers, and/or textile materials, including flexible and/or braided textiles. Textile materials can include memory-formed textiles. The shunt 302 and/or stent 304 may be configured to collapse to a smaller diameter for delivery while maintaining flexibility of the shunt 302 and/or stent 304. In some examples, the shunt 302 and/or stent 304 may be covered by a tubular sheath (not shown) configured to surround at least a portion of the shunt 302 and/or stent 304 and/or the shunt 302 and/or stent 304 may comprise a solid tubular material. The sheath may be configured to prevent the implant from expanding from a crimped configuration. In some examples, additional and/or alternative devices and/or methods may be used to prevent expansion of the shunt 302 and/or stent 304.

The shunt 302 may be situated at least partially between the first end 312 and the second end 314. The shunt 302 may form a channel and/or lumen through which blood can flow.

In some examples, the shunt 302 can comprise one or more flared barbs on lumen-facing surfaces of the flanges configured to stabilize the shunt 302 by anchoring the shunt 302 to the tissue wall(s). The distal flange and/or proximal flange may comprise one or more such flared barbs.

The shunt 302 may be at least partially composed of bare and/or enclosed metal and/or other material. In some examples, the shunt 302 may be bare in the case of adjacent anatomic structures (e.g., an adjacent RPA 13 and SVC 15) and/or the shunt 302 may be at least partially covered in the case of non-adjacent anatomic structures. For example, the at least partially covered shunt 302 may be configured to prevent infiltration of blood into the thoracic cavity and/or other anatomical area.

In some examples, a delivery process for delivering the shunt 302 and/or stent 304 may involve delivery through multiple blood vessels. For example, the internal jugular vein and the right femoral vein would be used for dual access. The shunt 302 and/or stent 304 may be placed at the end of a transcatheter delivery system which can traverse the right atrium, right ventricle, into the right pulmonary artery. This delivery catheter may or may not have an end hole or side hole catheter to inject contrast to confirm location. The catheter, in some examples, would have one or more articulation points at the distal end of the catheter to allow for manipulation and angulation, with a needle at the distal end for puncture. In some examples, there may be a loop or a snare marker in the SVC 15 to allow for targeted puncture, and/or capture of the distal end or wire as needed. The shunt 302 may be extended across the RPA-SVC to be placed and create a shunt between the RPA 13 to SVC 15.

In another example, a coiled wire, snare, or wire marker may be used to traverse the right atrium 5, right ventricle 4, and RPA 13, with imaging guidance. The imaging guidance may come in the form of a catheter with end or side hole contrast angiography, with a radio-opaque tip, marker bands, or an echogenic tip, or a combination of the above. This marker can mark the RPA 13 site. The delivery catheter can then be utilized in the SVC 15 from either the femoral vein or the internal jugular vein with one or more articulation points at the distal end of the delivery catheter to better facilitate targeted puncture of the SVC-RPA. With adjunct imaging guidance, the SVC 15 and RPA 13 may be punctured with subsequent placement of the device and creation of the shunt.

In some examples, the shunt 302 may be placed at the RPA/SVC junction to effectively reduce systolic and/or mean pulmonary artery pressure in patients while minimizing the impact on right ventricle function. While the RPA 13 and SVC 15 are described herein for illustration, the device may be a variable-orifice device.

The shunt 302 can comprise a valve shunt constructed of various metallic alloys and/or plastics. In some examples, the shunt 302 can include commissure posts configured to support the attachment of tissue leaflets. The shunt 302 can be preloaded to a certain force level while the leaflet assembly may be pre-formed such that the tissue leaflets may be naturally closed under a zero-pressure gradient and/or a small defined pressure gradient.

The leaflets may be hydraulically loaded to deform the shunt 302 such that the leaflets may be open to form an open orifice that allows blood flow. The leaflets can be configured to close when the pressure gradient drops below the set pressure or zero.

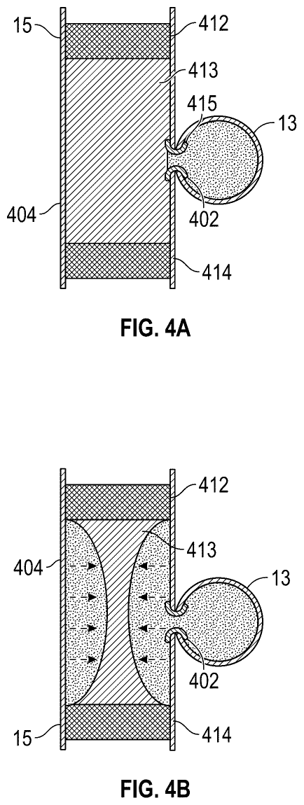

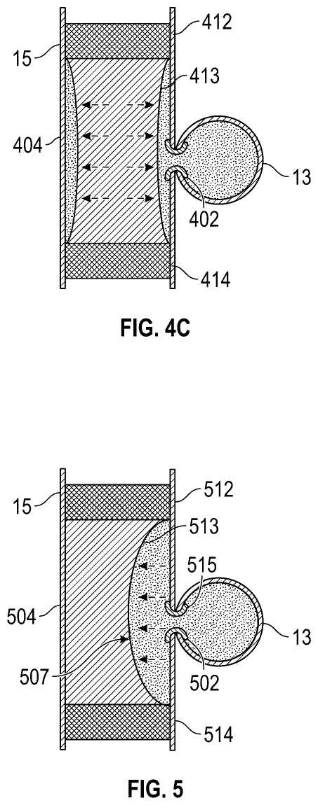

FIGS. 4A-4C illustrate an example shunting system for shunting blood between a first blood vessel and/or chamber (e.g., the RPA 13) and a second blood vessel and/or chamber (e.g., the SVC 15) in accordance with one or more examples. FIGS. 4A-4C provide cross-sectional side view of the SVC 15 and cross-sectional overhead views of the RPA 13. In some cases, the SVC 15 and the RPA 13 may extend generally orthogonally and/or perpendicularly to each other and/or may have a single intersection point.

In some examples, the system may comprise a shunt 402 configured to form and/or maintain a flow channel between the RPA 13 and the SVC 15 and/or between other blood vessels and/or chambers. In response to changing blood pressure, blood may flow through the shunt 402 (e.g., out of the RPA 13) and/or into contact with a stent 404 (e.g., into the SVC 15) and/or other implant disposed at least partially within the SVC 15. FIG. 4A illustrates a default and/or first state of the shunting system prior to movement of blood out of the RPA 13 into the SVC 15. FIG. 4B illustrates a second state of the shunting system in which blood pressure in the RPA 13 increases (e.g., during systole) a sufficient amount for excess blood to be pushed out of the RPA 13, through the shunt 402, and/or into the SVC 15. In some cases, blood pressure through the shunt 402 may be sufficiently high that the blood causes movement of the stent 404 and/or of a midsection 413 of the stent 404. FIG. 4C illustrates a third state of the shunting system in which the blood pressure in the RPA 13 decreases from the increased amount of the second state such that a recoil of the stent 404 causes a return to the shape and/or form shown in FIG. 4A to cause blood within the SVC 15 to move back through the shunt 402 and/or into the RPA 13. For example, the stent 404 (e.g., at least the midsection 413 of the stent 404) may be at least partially elastic and/or may be configured to elastically recoil back to an original and/or default form. The reduced pressure of the RPA 13 may allow spring-like materials of the stent 404 to push the blood back into the RPA 13 and/or towards the lungs.

In some examples, the stent 404 may be at least partially self-expanding and/or may be at least partially covered. The stent 404 and/or one or more components of the stent 404 may be sealed radially to prevent leakage of blood. For example, blood entering the SVC 15 via the shunt 402 may be sealed between a first end 412 of the stent 404 and a second end 414 of the stent 404. The first end 412 and/or the second end 414 may be configured to expand to a diameter and/or width that is equal to and/or greater than a diameter and/or width of the SVC 15. Accordingly, the first end 412 and/or the second end 414 may expand into contact with the walls of the SVC 15. In some examples, the first end 412 and/or the second end 414 may be configured to cause in-growth of tissue at the first end 412 and/or the second end 414 to improve a seal and/or anchoring of the first end 412 and/or the second end 414. For example, the first end 412 and/or the second end 414 may comprise a coating configured to facilitate in-growth of tissue around the first end 412 and/or the second end 414. In another example, the first end 412 and/or the second end 414 may comprise one or more barbs and/or needles configured to penetrate the surrounding tissue to promote in-growth of tissue around the first end 412 and/or the second end 414.

During systole, the pressure in the RPA 13 may rise above the pressure in the SVC 15, which may cause the stent 404 to at least partially compress. Compression of the stent 404 can add compliance to the pulmonary artery and/or reduce right ventricle afterload. During diastole, the pressure in the RPA 13 drops. As a result, a spring force of the stent 404 (e.g., shape-set components of the stent 404) can cause the stent 404 to expand radially and/or shunt blood back into the RPA 13 and into the lungs.

In some examples, the stent 404 may be at least partially covered. For example, at least an inner surface and/or outer surface of the midsection 413 of the stent 404 may comprise a frame at least partially enclosed by a covering. The covering can be made from various suitable materials, which can include one or more polymers, elastomers, and/or textiles.

The covering may not be totally fluid tight and/or may be at least partially permeable. In some examples, the covering may be configured to allow a controlled leakage of blood through the covering and/or into the SVC 15. Leakage of blood through the covering may prevent thrombus and/or shunt volume from the RPA 13 to the SVC 15. The covering may be configured to be at least partially fluid tight and/or may be configured to prevent a sufficient amount of leakage that the stent 404 may at least partially compress in response to trapped blood adjacent to the stent 404.

The first end 412 and/or second end 414 may comprise one or more covered and/or coated self-expanding materials. For example, the first end 412 and/or second end 414 may comprise self-expanding and/or balloon-expandable implants at least partially covered by one or more textiles and/or other suitable materials.

A puncture may be created in the SVC 15 and/or RPA 13 to accommodate the shunt 402. In some examples, the shunt 402 may be configured to extend at least partially into the SVC 15 and/or RPA 13 and/or across any gap between the SVC 15 and RPA 13. The shunt 402 may be configured to form a bridge between the SVC 15 and RPA 13 and/or may at least partially pull the SVC 15 towards the RPA 13 and/or the RPA 13 towards the SVC 15.

While the shunt 402 is described as bridging the SVC 15 and RPA 13, the shunt 402 and/or additional shunts may additionally or alternatively be used at other anatomical locations, including as a shunt between the RPA 13 and other venous side vessels and/or chambers (e.g., the right atrium).

In some examples, the shunt 402 may be at least partially fluid tight and/or may be configured to provide a channel for blood flow between the SVC 15 and RPA 13. For example, as blood pressure in the RPA 13 increases (e.g., during systole), blood may be pushed from the RPA 13 through the shunt 402 and/or to the SVC 15. As blood pressure in the RPA 13 decreases (e.g., during diastole), blood pushed into the SVC 15 from the RPA 13 may be pushed and/or suctioned back into the RPA 13 via the shunt 402.

The shunt 402 can have any suitable form and/or structure. In some examples, the shunt 402 may comprise a laser-cut hypotube and/or other device. The shunt 402 may have an at least partially cylindrical form and/or may comprise a lumen configured to allow blood flow through an outer frame of the shunt 402. In some examples, the shunt 402 may comprise flared ends 415 (e.g., flanges) configured to facilitate anchoring of the shunt 402 against the walls of the SVC 15 and/or RPA 13.

The shunt 402 may be delivered to a point of the SVC 15 that is adjacent and/or near the RPA 13 and/or to a point of the RPA 13 that is adjacent to and/or near the SVC 15. For example, a puncture may be made and/or the shunt 402 may be delivered to a point of overlap between the SVC 15 and the RPA 13.

In some examples, the stent 404 may be delivered via catheter. The stent 404 may comprise one or more self-expanding and/or balloon-expandable materials and/or may be configured to naturally expand outwardly in the absence of substantial inward forces. In some examples, the stent 404 may be at least partially composed of one or more shape-memory alloys, which can include Nitinol and/or similar materials. The materials of the stent 404 may be shape-set in a generally open form. For example, the stent 404 may comprise an inner lumen that is fully open in the default form of the stent 404. The stent 404 may comprise walls and/or sides formed from sheets and/or lines of material that may be configured to bend inwardly in response to outward pressure (e.g., blood pressure).

The stent 404 may comprise a midsection 413 disposed between a first end 412 and/or a second end 414. The first end 412 and/or second end 414 may be configured to securely anchor to surrounding tissue and/or may be configured to resist deformation. The midsection 413 may be configured to be loosely anchored and/or detached from the surrounding tissue to allow the midsection 413 to bend inwardly in response to external pressure. In some examples, the stent 404 may be configured to be delivered adjacent to the shunt 402 and/or adjacent to a puncture point between the SVC 15 and/or RPA 13. For example, the midsection 413 may be configured to extend across an opening and/or channel formed between the SVC 15 and RPA 13.

In some examples, the stent 404 may be configured for delivery into the SVC 15. However, the stent 404 may be configured for delivery into the RPA 13 and/or other blood vessel and/or heart chamber.

The stent 404 may have a generally cylindrical shape and/or may have other suitable shapes. In some examples, the stent 404 may comprise a frame composed of one or more shape-memory alloys and/or generally rigid materials. The stent 404 may additionally comprise a covering and/or skirt at least partially enclosing an outer and/or inner surface of the frame. The covering may be at least partially fluid tight and/or may be configured to catch and/or retain blood pressed into contact with the covering. In some examples, the covering may be configured to extend along the midsection 413, first end 412 and/or second end 414 of the stent 404.

Blood passing through the shunt 402 (e.g., from the RPA 13) may press against the covering and/or may cause the midsection 413 of the stent 404 to bow inwardly. The first end 412 and/or second end 414 may be configured to retain a default form and/or may be configured not to bow inwardly in response to blood pressure changes. Accordingly, blood may be retained around the midsection 413 until the blood is pressed and/or pulled back through the shunt 402 (e.g., to the RPA 13). In some examples, the covering may not be fully fluid tight and/or may be configured to allow some blood flow through the covering to advantageously prevent clot and/or thrombus formation due to stagnant blood.

The stent 404 (e.g., deformable device, deformable balloon, deformable stent, expandable balloon, expandable stent, etc.) may be configured to elastically deform in response to increases in blood flow and/or blood pressure. For example, the stent 404 may be configured to move and/or deform from a generally cylindrical form to an hourglass and/or inwardly bent form. The stent 404 may be configured to elastically and/or naturally return to the generally cylindrical form in response to reduction in blood flow and/or blood pressure.

The system may be configured to provide a fluid-tight seal and/or to prevent flow between the RPA 13 and SVC 15 outside of the stent 404 and/or shunt 402. The stent 404 and/or shunt 402 may comprise an anchoring frame and/or a fluid-tight and/or fluid impeding covering. The frame(s) may be stitched, sutured, and/or otherwise attached to the covering(s). The seal created by the system can promote filling and/or compression of the stent 404 and/or expandable device and/or can minimize unwanted changes in blood flow caused by excessive blood flow between the RPA 13 and SVC 15. The stent 404 and/or shunt 402 may be configured to flushly and/or tightly extend along and/or around the native tissue walls to minimize gaps between the system and the native tissue.

The stent 404 may be at least partially elastic and/or may have elasticity. In some examples, the stent 404 may be configured to store energy when the stent 404 is deformed from the default state to the compressed and/or expanded state. For example, as the stent 404 is deformed, the stent 404 may naturally store potential energy to cause the stent 404 to naturally move back to the default form upon removal of deformation forces. The stent 404 may extend at least partially into the SVC 15 and/or energy stored at the stent 404 may be stored at least partially within the SVC 15.

Deformation of the stent 404 may be configured to reduce and/or cause reduction of blood flow through the SVC 15. For example, blood flow from the RPA 13 into and/or against the stent 404 may cause the stent 404 to extend into and/or across the SVC 15, thereby at least partially impeding blood flow through the SVC 15. In some examples, the stent 404 may be configured to diametrically bifurcate the SVC 15. Thus, increased blood pressure in the RPA 13 may cause reduced blood flow in the SVC 15 through use of the system.

FIG. 5 provides a side view of another example shunting system for shunting blood between a first blood vessel and/or chamber (e.g., the RPA 13) and a second blood vessel and/or chamber (e.g., the SVC 15) in accordance with one or more examples. The system may comprise a shunt 502 configured to channel blood flow from the RPA 13 towards a midsection 513 of a stent 504 disposed within the SVC 15. The midsection 513 may be disposed between a first end 512 and/or a second end 514 of the stent 504. In some examples, the midsection 513 may be configured to at least partially compress in response to increased blood pressure flowing into the SVC 15 from the RPA 13. However, one or more portions of the midsection 513 may be configured not to compress in response to increased blood pressure. For example, the midsection 513 may be configured to compress at a first side 507 adjacent to the shunt 502 and/or RPA 13 and/or other sides of the midsection 513 may be configured to maintain a default shape in response to increased pressure. For example, the first side 507 may have a thinner and/or more flexible structure than other sides and/or portions of the midsection 513. Similarly, the first end 512 and/or second end 514 may not be configured to compress and/or move in response to increased blood pressure. For example, the first end 512 and/or second end 514 may comprise thicker materials and/or different materials than the first side 507 of the midsection 513.

In some examples, the shunt 502 may comprise flared ends 515 (e.g., flanges) configured to facilitate anchoring of the shunt 502 against the walls of the SVC 15 and/or RPA 13.

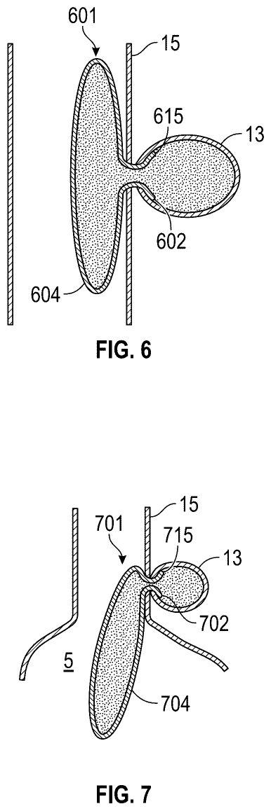

FIG. 6 illustrates an example compliance system 601 for shunting, transferring, and/or exchanging fluid (e.g., blood and/or saline) between a first blood vessel and/or chamber (e.g., the RPA 13) and a second blood vessel and/or chamber (e.g., the SVC 15) in accordance with one or more examples. The shunting system 601 may comprise a shunt 602 and/or shunt portion and/or an expandable and/or deformable device 604 and/or expandable portion. In some examples, the expandable device 604 may comprise a compliant bladder and/or vessel configured to be placed in the SVC 15 and/or right atrium. The expandable device 604 may be configured to add compliance to the pulmonary artery and/or to reduce right ventricle afterload. In some examples, the expandable device 604 may be configured to expand, compress, and/or deform in response to blood pressure. In some examples, the shunt 602 may be configured to temporarily transfer and/or exchange fluid (e.g., blood and/or saline) between the first blood vessel and/or chamber and the second blood vessel and/or chamber.

The expandable device 604 may be attached to the shunt 602 and/or extend from the shunt 602. However, the expandable device and the shunt 602 may be separate devices and/or may be disconnected in some examples. The expandable device 604 may be configured to receive blood flow from the RPA 13 via the shunt 602 and/or to expand in response to the blood flow. Additionally or alternatively, the device 604 may be configured to receive saline and/or other fluid contained within the shunt 602. For example, the shunt 602 may comprise a fluid-tight container containing saline and/or other fluid configured to move between a first portion of the shunt 602 disposed in the RPA 13 and/or a second portion of the shunt 602 (e.g., the device 604) disposed in the SVC 15. As the expandable device 604 expands, the expandable device 604 may be configured to extend further into the SVC 15 and/or to occupy an increased amount of the SVC 15. The shunt 602 may comprise a bladder and/or inflatable and/or expandable device disposed at least partially within the RPA 13 and/or configured to exchange fluid (e.g., saline) with the expandable device 604 (e.g., bladder).

In some examples, the expandable device 604 may be at least partially composed of one or more expandable and/or stretchy materials to allow a size and/or shape of the expandable device 604 to change in response to blood flow. The expandable device 604 may be configured to fold and/or otherwise compress in the absence of blood flow into the expandable device 604. In response to blood flow into the expandable device 604, the expandable device 604 may be configured to become taught and/or to stretch. The device 604 may be at least partially elastic and/or may have sufficient elasticity to generate recoil in response to inflation/expansion and/or to press fluid out of the device 604.

In some examples, the system may comprise a shunt 602 configured to form and/or maintain a flow channel between the RPA 13 and the SVC 15 and/or between other blood vessels and/or chambers. In response to changing blood pressure, blood may flow through the shunt 602 (e.g., out of the RPA 13) and/or into the expandable device 604 (e.g., into the SVC 15) and/or other implant disposed at least partially within the SVC 15.

In some examples, the expandable device 604 may be at least partially self-expanding and/or may be at least partially covered. For example, the expandable device 604 may comprise a frame composed at least partially of one or more shape-memory alloys (e.g., Nitinol) and/or the frame may be at least partially enclosed by one or more coverings. The covering(s) can be made from various suitable materials, which can include one or more polymers, elastomers, and/or textiles.

During systole, the pressure in the RPA 13 may rise above the pressure in the SVC 15, which may cause the expandable device 604 to at least partially inflate. Inflation of the expandable device 604 can add compliance to the pulmonary artery and/or reduce right ventricle afterload. During diastole, the pressure in the RPA 13 drops. As a result, a spring force of the expandable device 604 (e.g., shape-set components of the expandable device 604) can cause the expandable device 604 to compress and/or shunt blood back into the RPA 13 and into the lungs.

The covering may not be totally fluid tight. In some examples, the covering may be configured to allow a controlled leakage of blood through the covering and/or into the SVC 15. Leakage of blood through the covering may prevent thrombus and/or shunt volume from the RPA 13 to the SVC 15. The covering may be configured to be at least partially fluid tight and/or may be configured to prevent a sufficient amount of leakage that the expandable device 604 may at least partially compress in response to trapped blood adjacent to the expandable device 604.

A puncture may be created in the SVC 15 and/or RPA 13 to accommodate the shunt 602. In some examples, the shunt 602 may be configured to extend at least partially into the SVC 15 and/or RPA 13 and/or across any gap between the SVC 15 and RPA 13. The shunt 602 may be configured to form a bridge between the SVC 15 and RPA 13 and/or may at least partially pull the SVC 15 towards the RPA 13 and/or the RPA 13 towards the SVC 15.

While the shunt 602 is described as bridging the SVC 15 and RPA 13, the shunt 602 and/or additional shunts may additionally or alternatively be used at other anatomical locations, including as a shunt between the RPA 13 and other venous side vessels and/or chambers (e.g., the right atrium).

In some examples, the shunt 602 may be at least partially fluid tight and/or may be configured to provide a channel for blood flow between the SVC 15 and RPA 13. For example, as blood pressure in the RPA 13 increases (e.g., during systole), blood may be pushed from the RPA 13 through the shunt 602 and/or to the SVC 15. As blood pressure in the RPA 13 decreases (e.g., during diastole), blood pushed into the SVC 15 from the RPA 13 may be pushed and/or suctioned back into the RPA 13 via the shunt 602.

The shunt 602 can have any suitable form and/or structure. In some examples, the shunt 602 may comprise a laser-cut hypotube and/or other device. The shunt 602 may have an at least partially cylindrical form and/or may comprise a lumen configured to allow blood flow through an outer frame of the shunt 602. In some examples, the shunt 602 may comprise flared ends 615 (e.g., flanges) configured to facilitate anchoring of the shunt 602 against the walls of the SVC 15 and/or RPA 13.

The shunt 602 may be delivered to a point of the SVC 15 that is adjacent and/or near the RPA 13 and/or to a point of the RPA 13 that is adjacent to and/or near the SVC 15. For example, a puncture may be made and/or the shunt 602 may be delivered to a point of overlap between the SVC 15 and the RPA 13.

In some examples, the expandable device 604 may be delivered via catheter. The expandable device 604 may comprise one or more self-expanding and/or balloon-expandable materials and/or may be configured to naturally expand outwardly in the absence of substantial inward forces. In some examples, the expandable device 604 may be at least partially composed of one or more shape-memory alloys, which can include Nitinol and/or similar materials.

In some examples, the expandable device 604 may be configured for delivery into the SVC 15. However, the expandable device 604 may be configured for delivery into the RPA 13 and/or other blood vessel and/or heart chamber.

In some examples, the expandable device 604 may comprise a frame composed of one or more shape-memory alloys and/or generally rigid materials. The expandable device 604 may additionally comprise a covering and/or skirt at least partially enclosing an outer and/or inner surface of the frame. The covering may be at least partially fluid tight and/or may be configured to catch and/or retain blood pressed into contact with the covering.

Blood passing through the shunt 602 (e.g., from the RPA 13) may press against the covering and/or may cause the expandable device 604 to stretch outwardly. In some examples, the covering may not be fully fluid tight and/or may be configured to allow some blood flow through the covering to advantageously prevent clot and/or thrombus formation due to stagnant blood.

The system may be configured to provide a fluid-tight seal and/or to prevent flow between the RPA 13 and SVC 15 outside of the expandable device 604 and/or shunt 602. The expandable device 604 and/or shunt 602 may comprise an anchoring frame and/or a fluid-tight and/or fluid impeding covering. The frame(s) may be stitched, sutured, and/or otherwise attached to the covering(s). The seal created by the system can promote filling and/or compression of the expandable device 604 and/or expandable device and/or can minimize unwanted changes in blood flow caused by excessive blood flow between the RPA 13 and SVC 15. The expandable device 604 and/or shunt 602 may be configured to flushly and/or tightly extend along and/or around the native tissue walls to minimize gaps between the system and the native tissue.

The expandable device 604 may be at least partially elastic and/or may have elasticity. In some examples, the expandable device 604 may be configured to store energy when the expandable device 604 is deformed from the default state to the compressed and/or expanded state. For example, as the expandable device 604 is deformed, the expandable device 604 may naturally store potential energy to cause the expandable device 604 to naturally move back to the default form upon removal of deformation forces. The expandable device 604 may extend at least partially into the SVC 15 and/or energy stored at the expandable device 604 may be stored at least partially within the SVC 15.

Deformation of the expandable device 604 may be configured to reduce and/or cause reduction of blood flow through the SVC 15. For example, blood flow from the RPA 13 into and/or against the expandable device 604 may cause the expandable device 604 to extend into and/or across the SVC 15, thereby at least partially impeding blood flow through the SVC 15. In some examples, the expandable device 604 may be configured to diametrically bifurcate the SVC 15. Thus, increased blood pressure in the RPA 13 may cause reduced blood flow in the SVC 15 through use of the system.

FIG. 7 illustrates an example compliance system 701 for shunting, transferring, and/or exchanging fluid (e.g., blood and/or saline) between a first blood vessel and/or chamber (e.g., the RPA 13) and a second blood vessel and/or chamber (e.g., the SVC 15) in accordance with one or more examples. The shunting system 701 may comprise a shunt 702 and/or shunt portion and/or an expandable device 704 and/or expandable portion. In some examples, the expandable device 704 may comprise a compliant bladder and/or vessel configured to be placed in the SVC 15 and/or right atrium 5. The expandable device 704 may be configured to add compliance to the pulmonary artery and/or to reduce right ventricle afterload.

The expandable device 704 may be attached to the shunt 702 and/or extend from the shunt 702. However, the expandable device and the shunt 702 may be separate devices and/or may be disconnected in some examples. The expandable device 704 may be configured to receive blood flow from the RPA 13 via the shunt 702 and/or to expand in response to the blood flow. As the expandable device 704 expands, the expandable device 704 may be configured to extend further into the SVC 15 and/or to descend out of the SVC 15 and/or into the right atrium 5. In some examples, the expandable device 704 may be configured to extend deeper and/or further in a first direction (e.g., downward and/or towards the right atrium 5) than in a second direction (e.g., upward and/or away from the right atrium 5). For example, the expandable device 704 may be shape-set to promote expansion in a desired direction and/or may be configured to move in response to gravity and/or blood flow.

Expansion of the expandable device 704 into the right atrium 5 may advantageously afford greater expansion ability of the expandable device 704. For example, the right atrium 5 may provide greater volume and/or space for the expandable device 704 to fill. In this way, restriction from the SVC 15 and/or other anatomical features may be minimized. Moreover, expansion of the expandable device 704 out of the SVC 15 and/or into the right atrium 5 may minimize blockage and/or obstruction of the SVC 15 by the expandable device 704.

In some examples, the expandable device 704 may be at least partially composed of one or more expandable and/or stretchy materials to allow a size and/or shape of the expandable device 704 to change in response to blood flow. The expandable device 704 may be configured to fold and/or otherwise compress in the absence of blood flow into the expandable device 704. In response to blood flow into the expandable device 704, the expandable device 704 may be configured to become taught and/or to stretch.

In some examples, the system may comprise a shunt 702 configured to form and/or maintain a flow channel between the RPA 13 and the SVC 15 and/or between other blood vessels and/or chambers. In response to changing blood pressure, blood may flow through the shunt 702 (e.g., out of the RPA 13) and/or into the expandable device 704 (e.g., into the SVC 15) and/or other implant disposed at least partially within the SVC 15.

In some examples, the expandable device 704 may be at least partially self-expanding and/or may be at least partially covered. For example, the expandable device 704 may comprise a frame composed at least partially of one or more shape-memory alloys (e.g., Nitinol) and/or the frame may be at least partially enclosed by one or more coverings. The covering(s) can be made from various suitable materials, which can include one or more polymers, elastomers, and/or textiles.

During systole, the pressure in the RPA 13 may rise above the pressure in the SVC 15, which may cause the expandable device 704 to at least partially inflate. Inflation of the expandable device 704 can add compliance to the pulmonary artery and/or reduce right ventricle afterload. During diastole, the pressure in the RPA 13 drops. As a result, a spring force of the expandable device 704 (e.g., shape-set components of the expandable device 704) can cause the expandable device 704 to compress and/or shunt blood back into the RPA 13 and into the lungs. The expandable device 704 may be at least partially elastic and/or may be configured to store potential energy in response to deformation (e.g., caused by increased blood pressure and/or flow into and/or around the expandable device 704). Accordingly, the device 704 may be configured to convert increased blood flow in the RPA 13 to stored energy and/or increase compliance in the SVC 15 and/or other blood vessel.

The covering may not be fully fluid tight. In some examples, the covering may be configured to allow a controlled leakage of blood through the covering and/or into the SVC 15. Leakage of blood through the covering may prevent thrombus and/or shunt volume from the RPA 13 to the SVC 15. The covering may be configured to be at least partially fluid tight and/or may be configured to prevent a sufficient amount of leakage that the expandable device 704 may at least partially compress in response to trapped blood adjacent to the expandable device 704.

A puncture may be created in the SVC 15 and/or RPA 13 to accommodate the shunt 702. In some examples, the shunt 702 may be configured to extend at least partially into the SVC 15 and/or RPA 13 and/or across any gap between the SVC 15 and RPA 13. The shunt 702 may be configured to form a bridge between the SVC 15 and RPA 13 and/or may at least partially pull the SVC 15 towards the RPA 13 and/or the RPA 13 towards the SVC 15.

While the shunt 702 is described as bridging the SVC 15 and RPA 13, the shunt 702 and/or additional shunts may additionally or alternatively be used at other anatomical locations, including as a shunt between the RPA 13 and other venous side vessels and/or chambers (e.g., the right atrium 5).

In some examples, the shunt 702 may be at least partially fluid tight and/or may be configured to provide a channel for blood flow between the SVC 15 and RPA 13. For example, as blood pressure in the RPA 13 increases (e.g., during systole), blood may be pushed from the RPA 13 through the shunt 702 and/or to the SVC 15. As blood pressure in the RPA 13 decreases (e.g., during diastole), blood pushed into the SVC 15 from the RPA 13 may be pushed and/or suctioned back into the RPA 13 via the shunt 702.

The shunt 702 can have any suitable form and/or structure. In some examples, the shunt 702 may comprise a laser-cut hypotube and/or other device. The shunt 702 may have an at least partially cylindrical form and/or may comprise a lumen configured to allow blood flow through an outer frame of the shunt 702. In some examples, the shunt 702 may comprise flared ends 715 (e.g., flanges) configured to facilitate anchoring of the shunt 702 against the walls of the SVC 15 and/or RPA 13.

The shunt 702 may be delivered to a point of the SVC 15 that is adjacent and/or near the RPA 13 and/or to a point of the RPA 13 that is adjacent to and/or near the SVC 15. For example, a puncture may be made and/or the shunt 702 may be delivered to a point of overlap between the SVC 15 and the RPA 13.

In some examples, the expandable device 704 may be delivered via catheter. The expandable device 704 may comprise one or more self-expanding and/or balloon-expandable materials and/or may be configured to naturally expand outwardly in the absence of substantial inward forces. In some examples, the expandable device 704 may be at least partially composed of one or more shape-memory alloys, which can include Nitinol and/or similar materials.

The expandable device 704 (e.g., deformable device, deformable balloon, deformable stent, expandable balloon, expandable stent, etc.) may be configured to elastically deform in response to increases in blood flow and/or blood pressure (e.g., in the RPA 13). For example, the expandable device 704 may be configured to move and/or deform from a smaller and/or deflated form to an inflated and/or spherical and/or oval-shaped form. The expandable device 704 may be configured elastically and/or naturally return to the smaller and/or deflated form in response to a reduction in blood flow and/or blood pressure (e.g., in the RPA 13).

In some examples, the expandable device 704 may be configured for delivery into the SVC 15. However, the expandable device 704 may be configured for delivery into the RPA 13 and/or other blood vessel and/or heart chamber.

In some examples, the expandable device 704 may comprise a frame composed of one or more shape-memory alloys and/or generally rigid materials. The expandable device 704 may additionally comprise a covering and/or skirt at least partially enclosing an outer and/or inner surface of the frame. The covering may be at least partially fluid tight and/or may be configured to catch and/or retain blood pressed into contact with the covering.

Blood passing through the shunt 702 (e.g., from the RPA 13) may press against the covering and/or may cause the expandable device 704 to stretch outwardly. In some examples, the covering may not be fully fluid tight and/or may be configured to allow some blood flow through the covering to advantageously prevent clot and/or thrombus formation due to stagnant blood.

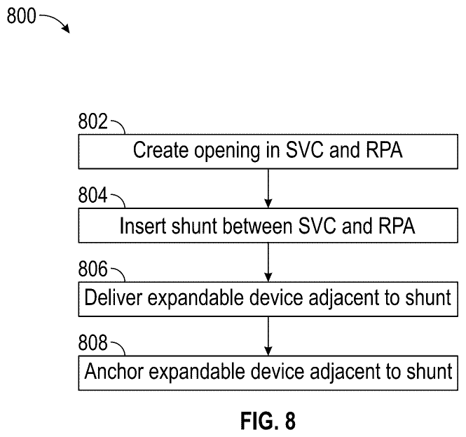

FIG. 8 provides a flowchart illustrating an example process 800 for delivery of one or more shunt implant and/or shunt systems describe herein. Steps of the process 800 may be performed in any suitable order and/or step(s) may me added and/or removed as needed.

At step 802, the process 800 involves creating an opening in an SVC and/or RPA. In some examples, a first opening in the SVC may be created at a point in a wall of the SVC that crosses and/or overlaps with the RPA. Similarly, a second opening in the RPA may be created at a point in a wall of the RPA that crosses and/or overlaps with the SVC. The SVC and/or RPA may be accessed by any suitable means and/or the opening(s) may be created in a single step or multiple steps.

At step 804, the process 800 involves inserting a shunt through the created opening(s) and/or extending the shunt between the SVC and the RPA. In some examples, the shunt may be configured to be disposed at least partially within the SVC and/or at least partially within the RPA. The shunt may extend across any gap between the SVC and RPA. In some examples, the shunt may comprise flared ends and/or various anchoring features to facilitate anchoring of the shunt in place.

At step 806, the process 800 involves delivering an expandable device and/or implant adjacent to the shunt. In some examples, the expandable device may be delivered to the SVC. Alternatively, the expandable device may be delivered to the RPA.

The expandable device can comprise a self-expandable and/or balloon-expandable stent and/or similar device and/or may comprise an inflatable and/or expandable balloon and/or similar device. The expandable device may have a cylindrical, spherical, oval-shaped, and/or other suitable form. The expandable device may be configured to be inflated and/or compressed by blood flow passing through the shunt. In some examples, the expandable device may be configured to generally prevent blood flow from the RPA from escaping beyond the expandable device. For example, the expandable device may be configured to retain a majority of blood passing through the shunt within the expandable device (e.g., as an inflatable balloon). In another example, the expandable device may be configured to retain a majority of blood passing through the shunt around a midsection of the expandable device and/or the expandable device may comprise end portions configured to substantially prevent blood flow above or below the expandable device. In some examples, the expandable device may be at least partially porous and/or may be configured to allow at least partial blood leakage through the expandable device.

At step 808, the process 800 involves anchoring the expandable device adjacent to the shunt and/or adjacent to the opening created in the SVC and/or RPA. In some examples, the expandable device may be configured to attach to the shunt. For example, the expandable device and/or shunt may comprise one or more hooks configured to mate with corresponding hooks and/or features of the expandable device and/or shunt. However, the expandable device may be configured to independently anchor adjacent to the shunt. In some examples, the expandable device may comprise one or more anchoring elements, which can include hooks, needles, pins, and/or sealing materials. The expandable device may be configured to expand into contact with the surrounding tissue walls to securely anchor the expandable device in place.

The expandable device may be anchored on either side and/or on both sides of the shunt. For example, the shunt may be configured to feed blood flow towards a midsection of the expandable device and/or the expandable device may comprise a first end and/or a second end on either side and/or on both sides of the midsection configured to substantially trap and/or contain blood at or near the midsection and/or between the first end and the second end. Delivery systems (e.g., catheters) may be removed from the body while the shunt and/or expandable device remain anchored.

### ADDITIONAL DESCRIPTION OF EXAMPLES

Provided below is a list of examples, each of which may include aspects of any of the other examples disclosed herein. Furthermore, aspects of any example described above may be implemented in any of the numbered examples provided below. Described herein are various example medical sheaths and/or delivery methods. Some examples described herein may be used in combination and/or may be used independently.

Example 1: A shunt system comprising: a shunt configured to maintain a blood flow pathway between a first blood vessel and a second blood vessel; and a deformable device configured to elastically deform in response to blood flow through the shunt.

Example 2: The shunt system of any example herein, in particular example 1, wherein the shunt is detached from the deformable device.

Example 3: The shunt system of any example herein, in particular example 1, wherein the shunt is configured to attach to the deformable device.

Example 4: The shunt system of any example herein, in particular example 1, wherein the deformable device comprises a stent.

Example 5: The shunt system of any example herein, in particular example 4, wherein the stent is at least partially enclosed by a covering.

Example 6: The shunt system of any example herein, in particular example 5, wherein the covering is substantially fluid tight.

Example 7: The shunt system of any example herein, in particular example 6, wherein the covering is configured to allow some blood flow through the covering.

Example 8: The shunt system of any example herein, in particular example 4, wherein the stent has a cylindrical form.

Example 9: The shunt system of any example herein, in particular example 4, wherein the stent comprises a midsection disposed between a first end and a second end.

Example 10: The shunt system of any example herein, in particular example 9, wherein the midsection is configured to bend inwardly in response to increased blood pressure through the shunt.

Example 11: The shunt system of any example herein, in particular example 10, wherein the midsection is configured to return to a default form in response to decreased blood pressure through the shunt.

Example 12: The shunt system of any example herein, in particular example 9, wherein the first end is at least partially enclosed by a sealing material.

Example 13: The shunt system of any example herein, in particular example 12, wherein the sealing material is configured to promote in-growth of tissue.

Example 14: The shunt system of any example herein, in particular example 9, wherein the first end is configured not to deform in response to blood flow through the shunt.

Example 15: The shunt system of any example herein, in particular example 9, wherein the second end is configured not to deform in response to blood flow through the shunt.

Example 16: The shunt system of any example herein, in particular example 9, wherein midsection is configured to elastically deform on a first side and not elastically deform on a second side opposite the first side.

Example 17: The shunt system of any example herein, in particular example 1, wherein the deformable device is configured to inflate with blood flow through the shunt.

Example 18: The shunt system of any example herein, in particular example 1, wherein the deformable device is oval-shaped.

Example 19: A method comprising: percutaneously delivering a shunt into a first blood vessel near an intersection point between the first blood vessel and a second blood vessel; passing the shunt at least partially through a vessel wall of the first blood vessel until the shunt enters the second blood vessel; and percutaneously delivering a deformable device into the first blood vessel near the intersection point between the first blood vessel and the second blood vessel, the deformable device configured to elastically deform in response to blood flow through the shunt.

Example 20: The method of any example herein, in particular example 19, wherein the shunt is detached from the deformable device.

Example 21: The method of any example herein, in particular example 19, wherein the shunt is configured to attach to the deformable device.

Example 22: The method of any example herein, in particular example 19, wherein the deformable device comprises a stent.

Example 23: The method of any example herein, in particular example 22, wherein the stent is at least partially enclosed by a covering.

Example 24: The method of any example herein, in particular example 23, wherein the covering is substantially fluid tight.

Example 25: The method of any example herein, in particular example 24, wherein the covering is configured to allow some blood flow through the covering.

Example 26: The method of any example herein, in particular example 22, wherein the stent comprises a midsection disposed between a first end and a second end.

Example 27: The method of any example herein, in particular example 26, wherein the midsection is configured to bend inwardly in response to increased blood pressure through the shunt.

Example 28: The method of any example herein, in particular example 26, wherein midsection is configured to elastically deform on a first side and not elastically deform on a second side opposite the first side.

Example 29: The method of any example herein, in particular example 19, wherein the deformable device is configured to inflate with blood flow through the shunt.

Example 30: The method of any example herein, in particular example 19, wherein the deformable device is oval-shaped.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

Delivery systems as described herein may be used to position catheter tips and/or catheters to various areas of a human heart. For example, a catheter tip and/or catheter may be configured to pass from the right atrium into the coronary sinus. However, it will be understood that the description can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels, and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device comprising an inner lumen configured to slidably receive instrumentation, such as for positioning within an atrium or coronary sinus, including for example delivery catheters and/or cannulas. It will be understood that other types of medical implant devices and/or procedures can be delivered to the coronary sinus using a delivery system as described herein, including for example ablation procedures, drug delivery and/or placement of coronary sinus leads.

What is claimed is:

1. A method of adding vascular compliance, the method comprising:
creating a first opening in a right pulmonary artery;
creating a second opening in a superior vena cava, wherein the second opening is aligned with the first opening;
percutaneously delivering a shunt into the first opening and the second opening, wherein the shunt extends into the right pulmonary artery and the superior vena cava; and
percutaneously delivering a stent into the superior vena cava adjacent to the shunt to add vascular compliance to one or both of the right pulmonary artery and the superior vena cava, the stent comprising a covering to at least partially prevent flow of blood from the shunt through the stent, the stent being at least partially flexible to allow the stent to bend away from walls of the superior vena cava in response to inflow of blood through the shunt, and the stent being sized to seal against the superior vena cava at a first end and a second end of the stent to trap blood between the stent and the walls of the superior vena cava.

2. The method of claim 1, wherein the first end of the stent comprises a coating to cause in-growth of tissue at the first end of the stent.

3. The method of claim 1, wherein the first end of the stent comprises one or more barbs or needles to penetrate the superior vena cava and promote in-growth of tissue at the first end.

4. The method of claim 1, wherein the stent is at least partially self-expanding.

5. The method of claim 1, further comprising attaching the stent to the shunt.

6. The method of claim 1, wherein the stent comprises a first side and a second side, the method further comprising percutaneously delivering the first side of the stent adjacent to the shunt, wherein the first side is flexible to allow the first side to bend away from the walls of the superior vena cava, and wherein the second side is less flexible than the first side to resist bending of the second side.

7. The method of claim 1, wherein the stent has a non-circular cross-sectional shape.

8. A method of adding vascular compliance, the method comprising:
creating a first opening in a first blood vessel;
creating a second opening in a second blood vessel, wherein the second opening is aligned with the first opening;
percutaneously delivering a shunt into the first opening and the second opening; and
percutaneously delivering a stent into the first blood vessel adjacent to the shunt to add vascular compliance to one or both of the first blood vessel and the second blood vessel, the stent comprising a covering to at least partially prevent flow of blood from the shunt through the stent, the stent being at least partially flexible to allow the stent to bend away from walls of the first blood vessel in response to inflow of blood through the shunt, and the stent being sized to seal against the first blood vessel at a first end and a second end of the stent to trap inflow of blood via the shunt between the stent and the walls of the first blood vessel.

9. The method of claim 8, wherein the first blood vessel is a superior vena cava and the second blood vessel is a right pulmonary artery.

10. The method of claim 8, wherein the shunt extends into the first blood vessel and the second blood vessel.

11. The method of claim 8, wherein the first end of the stent comprises a coating to cause in-growth of tissue at the first end of the stent.

12. The method of claim 8, wherein the first end of the stent comprises one or more barbs or needles to penetrate the first blood vessel and promote in-growth of tissue at the first end.

13. The method of claim 8, wherein the stent is at least partially self-expanding.

14. The method of claim 8, further comprising attaching the stent to the shunt.

15. The method of claim 8, wherein the stent comprises a first side and a second side, the method further comprising percutaneously delivering the first side of the stent adjacent to the shunt, wherein the first side is flexible to allow the first side to bend away from the walls of the first blood vessel, and wherein the second side is less flexible than the first side to resist bending of the second side.

16. A method of adding vascular compliance, the method comprising:
creating a first opening in a first blood vessel;
creating a second opening in a second blood vessel, wherein the second opening is aligned with the first opening;
percutaneously delivering a shunt into the first opening and the second opening; and
percutaneously delivering a compliant bladder into the first blood vessel adjacent to the shunt to add vascular compliance to one or both of the first blood vessel and the second blood vessel, the compliant bladder being inflatable to receive flow of fluid from the shunt in response to blood pressure changes in the first blood vessel or second blood vessel and being elastic to press fluid from the compliant bladder into the shunt in response to blood pressure changes in the first blood vessel or second blood vessel.

17. The method of claim 16, wherein the first blood vessel is a superior vena cava and the second blood vessel is a right pulmonary artery.

18. The method of claim 16, wherein the shunt extends into the first blood vessel and the second blood vessel.

19. The method of claim 16, further comprising attaching the compliant bladder to the shunt.

20. The method of claim 16, wherein the compliant bladder comprises a frame enclosed by a covering.
